(19) United States
(12) Patent Application Publication
(10) Pub. No.: US 2018/0221204 A1
(43) Pub. Date: Aug. 9, 2018

(54) SYSTEM AND METHOD FOR CONTACT LENS INSERTION AND REMOVAL

(71) Applicant: TRINADY SCHERIE MOORE, Reno, NV (US)

(72) Inventor: TRINADY SCHERIE MOORE, Reno, NV (US)

(21) Appl. No.: 15/889,333

(22) Filed: Feb. 6, 2018

Related U.S. Application Data
(60) Provisional application No. 62/455,313, filed on Feb. 6, 2017.

(51) Int. Cl. A61F 9/00

(52) U.S. Cl. CPC A61F 9/0061; A61F 2240/001

(57) ABSTRACT

A system may include a contact lens, a positioning member for positioning the contact lens, and a bead of adhesive material adhering the contact lens to the positioning member.

### FIELD OF THE DISCLOSURE

This disclosure relates generally to optical device application and more specifically to systems and methods for contact lens insertion and removal.

### BACKGROUND

A contact lens wearer typically uses their finger to insert and remove contact lenses. Insertion of a contact lens may include pushing the wetted lens onto the eye until it adheres while removal of a contact lens typically includes lightly pinching the eye to disengage the contact lens and then pulling the lens from the eye. The pressure applied to the eye may result in pain and discomfort to the wearer. Those who are new to wearing contact lenses and children may be particularly intimidated at the prospect of touching and pinching their eyes. Further, using fingers to remove contact lenses may be unsanitary as there is a high likelihood of introducing bacteria or other microbes into the eye, which increases the chance of infection. Likewise, fingers may include residues, such as from lotions or other chemicals that can cause pain and watering of the eye. Also, contact lens wearers may be prevented from wearing long fingernails or artificial fingernails in order to avoid scratching their eyes while inserting or removing contact lenses.

Some systems have been developed that rely on a suction cup to adhere to the contact lens. However, these systems often result in an uncomfortable pulling sensation on the eye and may result in too much pressure being placed thereon. Likewise, a system using tweezers has been developed. However, as with fingers the tweezers are used to lightly pinch the eye, which may result in discomfort.

Other systems have been developed for positioning jewels or stones using beaded wax. However, these systems typically rely on a wax that is hard and typically unsuitable for adherence to contact lenses. Further, these systems may be toxic or otherwise harmful to an eye.

### SUMMARY

Described are systems and methods for easily inserting and removing contact lenses from an eye without using fingers. The systems and methods rely on an adhesive material, such as jelly or wax, attached to the end of a positioning member. The adhesive material may be touched to a contact lens causing the positioning member to temporarily stick to the contact lens. The contact lens may then easily be positioned within the eye using the positioning member. Gently tapping and rotating the positioning member away from the lens may disengage the contact from the adhesive material, leaving the lens on the eye. For removal of the contact lens, the adhesive material may be touched to the lens while the lens is in the eye causing the positioning member to again temporarily stick to the contact lens. The lens may then be swiped or slid out of eye by use of a gentle sliding or swiping motion.

In an embodiment, a system includes a contact lens, a positioning member for positioning the contact lens, and a bead of adhesive material adhering the contact lens to the positioning member.

In some embodiments, the positioning member is a straight wand. In some embodiments, the positioning member includes a support structure encased within the bead of adhesive. In some embodiments, the support structure has a bulb shape. In some embodiments, a surface of the support structure includes a porous texture. In some embodiments, the positioning member is made from wood, plastic, metal, or a combination thereof. In some embodiments, the adhesive material includes a wax material, a gel material, medical grade silicone, or combinations thereof. In some embodiments, a surface of the bead of adhesive material includes a concave indentation therein. In some embodiments, the indentation conforms to a surface of the contact lens.

In an embodiment, a method includes adhering a positioning member to a contact lens using a bead of adhesive material. The method further includes positioning the contact lens onto an eye while the positioning member is adhered to the contact lens. The method also includes disengaging the positioning member from the contact lens by applying at least a pulling or tapping motion to the positioning member.

In some embodiments, the method includes supporting the bead of adhesive at a support structure of the positioning member while positioning the contact lens onto the eye, the support structure encased within the bead of adhesive material. In some embodiments, the method includes receiving the contact lens into a concave indentation in a surface of the bead of adhesive material. In some embodiments, the method includes re-adhering the positioning member to the contact lens using the bead of soft wax while the contact lens is positioned on the eye, removing the contact lens from the eye while the positioning member is adhered to the contact lens, and disengaging the positioning member from the contact lens.

In an embodiment, a method includes forming a positioning member with a support structure having a bulb shape. The method further includes forming a bead of adhesive material on the positioning member, the bead of adhesive material encasing the bulb shape. The method also includes forming an indentation in a surface of the bead of adhesive material.

In some embodiments, the method includes forming a porous texture on the support structure before forming the bead of adhesive material on the positioning member. In some embodiments, the adhesive material is formed from a wax material, a gel material, medical grade silicone, or a combination thereof.

Because the disclosed systems and methods rely on a soft and pliable adhesive to retain a contact lens, discomfort during insertion and removal is reduced as compared to current methods. Further, a contact lens wearer's fingers are not introduced onto the eye, reducing the likelihood of infection or pain from residue.

### BRIEF DESCRIPTION OF THE DRAWINGS

FIG. 1 depicts an embodiment of a system for contact lens insertion and removal.

FIG. 2 depicts an embodiment of the system for contact lens insertion and removal.

FIG. 3 depicts an embodiment the system for contact lens insertion and removal.

FIG. 4 depicts an embodiment of a method for contact lens insertion.

FIG. 5 depicts an embodiment of a method for contact lens removal.

FIG. 6 depicts an embodiment of a method for forming a system for contact lens insertion and removal.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the scope of the disclosure.

### DETAILED DESCRIPTION

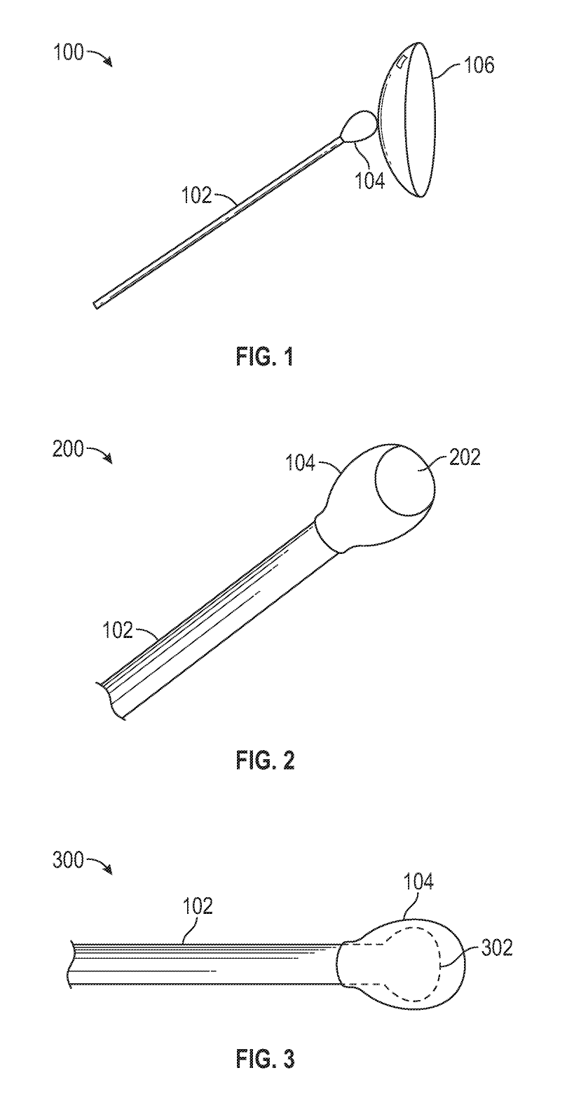

FIG. 1 depicts an embodiment of a system 100 for contact lens insertion and removal. The system 100 includes a positioning member 102, a bead of adhesive material 104, and a contact lens 106.

The positioning member 102 may be lightweight and durable, and may be used for multiple insertions and removals of the contact lens 106. Although FIG. 1 depicts the positioning member 102 as a straight wand, in some embodiments, it may have a curved shape. For example, the positioning member 102 may be molded into an ergonomic shape for the benefit of a user of the system 100. The positioning member 102 may include or be made from paper, wood, plastic, or metal. For example, the positioning member 102 may include a dowel formed from wood or heavy rolled paper. Alternatively, the positioning member 102 may include a material that is easily sterilized, such as plastic or aluminum. Regardless of the material, the positioning member 102 may be rigid enough to enable precise positioning of the contact lens 106. In some embodiments, the positioning member 102 may be hollow to result in a better joint with the bead of adhesive material 104 and to reduce production costs.

The bead of adhesive material 104 may be soft enough to provide a cushion that absorbs excess pressure applied to the contact lens 106 by the positioning member 102 when the contact lens 106 is pressed against an eye during insertion. The adhesive material 104 may include a soft wax, such as beeswax, paraffin wax, low melting microcrystalline wax, low-melting natural wax, or combinations thereof. Additives, such as solid starch fillers may be used to adjust the tackiness or adhesive properties of the wax. Alternatively, or in addition, the bead of adhesive material 104 may include a gel, vinyl, or rubber. Another material that may be used in the adhesive material is medical grade silicone. Because it may come into contact with an eye, the bead of adhesive material 104 may be non-toxic.

It should be noted that the positioning member 102 and the bead of adhesive material 104 may be packaged together and preassembled. Alternatively, the positioning member 102 and the adhesive material 104 may be packaged and sold separately, to be assembled by a consumer before use.

During contact lens insertion, the positioning member 102 may be adhered to the contact lens 106 using the bead of adhesive material 104. The contact lens 106 may then be positioned onto an eye while the positioning member 102 is adhered to the contact lens 106. After the contact lens 106 is applied to the eye, the positioning member 102 may be disengaged from the contact lens 106 by applying at least a tapping motion to the positioning member 102. For example, the contact lens 106 may be tapped to the eye with an associated blink to disengage the contact lens 106 from the positioning member 102. In some cases, a pulling motion may also be used and accompanied by a twisting or sliding motion to disengage the positioning member 102 from the contact lens 106. The motion may further be associated with a blink to disengage the positioning member 102.

During contact lens removal, the positioning member 102 may be adhered to the contact lens 106 using the bead of adhesive material 104 while the contact lens 106 is positioned on an eye. The adherence may be the result of tapping the bead of adhesive material 104 to an edge of the contact lens 106. The contact lens 106 may then be slid and turned simultaneously using the positioning member 102 to remove the contact lens 106 from the eye without much pressure and without discomfort. After the contact lens 106 is removed from the eye, the positioning member 102 may be disengaged from the contact lens 106 by applying at least a pulling, sliding, or rotating motion to the positioning member 102 while the contact lens 106 is retained within a case (not shown).

A benefit of the system 100 is that insertion and removal of a contact lens may be easier than current methods of insertion and removal. For example, using the system 100 a contact lens may be inserted in approximately 3 seconds as opposed to typical contact lens insertion methods, which may take longer. Another benefit of the system 100 is that contact lens insertion and removal may cause much less discomfort as compared to systems that rely other mechanisms, such as fingers, tweezers, or a suction cup. The system 100 may further be more sanitary than traditional systems for insertion and removal of contact lenses. Other advantages may exist.

Referring to FIG. 2, an embodiment of a system 200 for contact lens insertion and removal is depicted. The system 200 may correspond to the system 100. In the system 200, a surface of the bead of adhesive material 104 may have a concave indentation 202 therein. The concave indentation may conform to a surface of the contact lens 106.

An advantage of the concave indentation 202 is that it may enable more surface area contact between the bead of adhesive material 104 and the contact lens 106. By creating more surface area contact, adherence between the positioning member 102 and the contact lens 106 may be strengthened. Other advantages may exist.

Referring to FIG. 3, an embodiment of a system 300 for contact lens insertion and removal is depicted. The system 300 may correspond to the system 100. In the system 300, the positioning member 102 may include a support structure 302 encased within the adhesive material 104. The support structure 302 may include a lip or bulb shape. Further, a surface of the support structure 302 may have a porous texture in order to adhere better to the bead of adhesive material 104.

An advantage of the support structure 302 is that the structure and shape of the bead of adhesive material 104 may be preserved after multiple uses. Another advantage is that less adhesive material may be used during manufacturing, thereby resulting in lower production costs. Other advantages may exist.

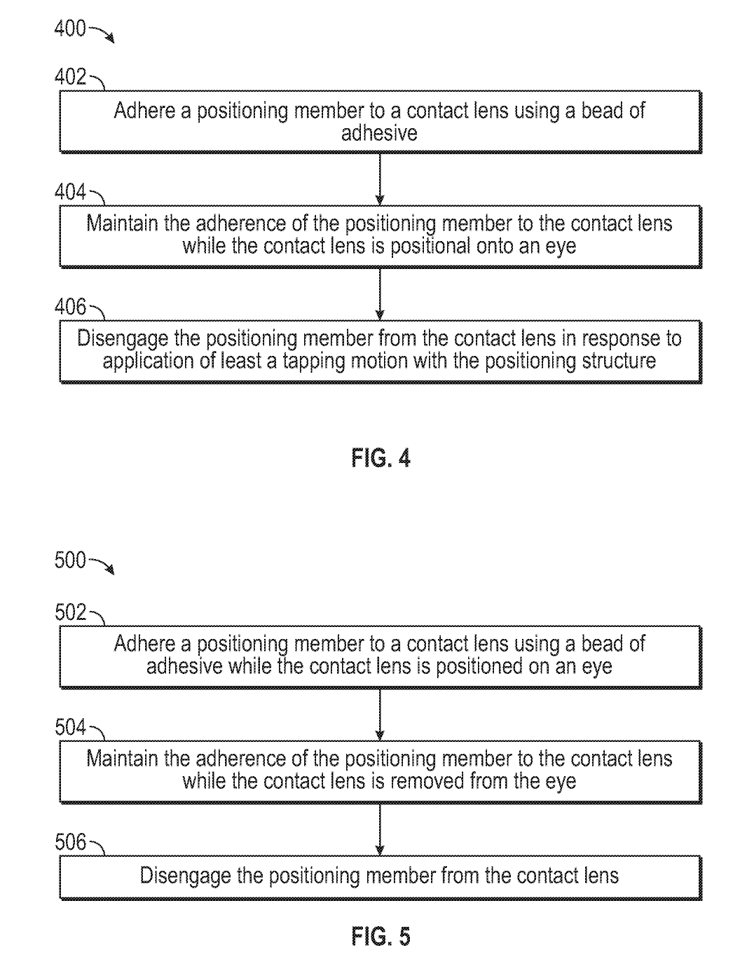

Referring to FIG. 4, an embodiment of a method 400 for contact lens insertion is depicted. The method 400 includes adhering a positioning member to a contact lens using a bead of adhesive material, at 402. For example, the positioning member 102 may be adhered to the contact lens 106.

The method 400 further includes maintaining the adherence of the positioning member to the contact lens while the contact lens is positioned onto an eye, at 404.

The method 400 also includes disengaging the positioning member from the contact lens in response to application of at least a tapping motion with the positioning member, at 406.

Referring to FIG. 5, an embodiment of a method 500 for contact lens removal is depicted. The method 500 includes adhering a positioning member to a contact lens using a bead of adhesive material while the contact lens is positioned on an eye, at 502. For example, the positioning member 102 may be adhered, or re-adhered, to the contact lens 106.

The method 500 further includes maintaining the adherence of the positioning member to the contact lens while the contact lens is removed from the eye.

The method 500 also includes disengaging the positioning member from the contact lens, at 506. For example, the positioning member 102 may be disengaged from the contact lens 106.

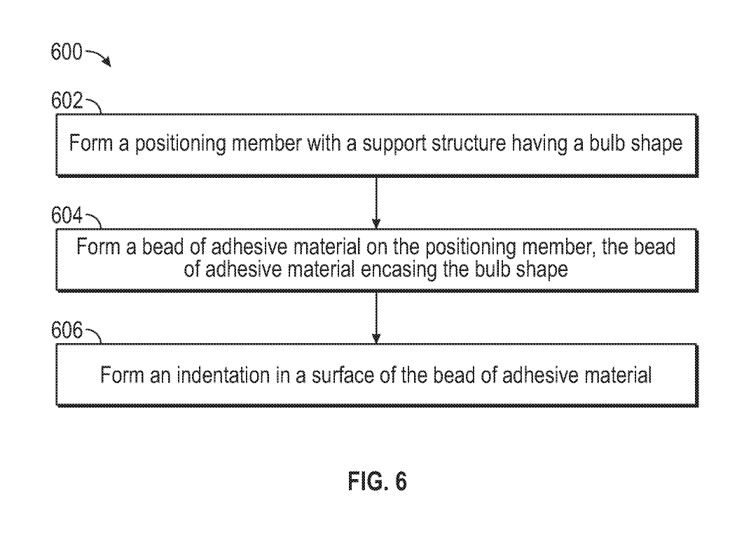

Referring to FIG. 6, an embodiment of a method 600 for forming a system for contact lens insertion and removal is depicted. The method 600 may include forming a positioning member with a support structure having a bulb shape, at 602. For example, the positioning member 102 may be formed with the support structure 302.

The method 600 further includes forming a bead of adhesive material on the positioning member, the bead of adhesive material encasing the bulb shape, at 604. For example, the bead of adhesive material 104 may be formed on the positioning member 102.

The method 600 also includes forming an indentation in a surface of the bead of adhesive material, at 606. For example, the indentation 202 may be formed in a surface of the bead of adhesive material 104.

A benefit of the method 600 is that by forming a positioning member that has a bulb shape, less adhesive material may be used to form a contact lens insertion and removal system. Further, by forming an indentation in a bead of adhesive material, more adhesion between a positioning member and a contact lens may be created. Other advantages may exist.

Although various embodiments have been shown and described, the present disclosure is not so limited and will be understood to include all such modifications and variations as would be apparent to one skilled in the art.

What is claimed is:

1. A system comprising:
a positioning member for positioning a contact lens; and
a bead of adhesive material for adhering the contact lens to the positioning member.

2. The system of claim 1, further comprising the contact lens adhered to the positioning member.

3. The system of claim 1, wherein the positioning member is a straight wand.

4. The system of claim 1, wherein the positioning member includes a support structure encased within the bead of adhesive, the support structure having a bulb shape.

5. The system of claim 4, wherein a surface of the support structure includes a porous texture.

6. The system of claim 1, wherein the positioning member is made from wood, plastic, metal, or a combination thereof.

7. The system of claim 1, wherein the adhesive material includes a wax material.

8. The system of claim 1, wherein the adhesive material includes a gel material.

9. The system of claim 1, wherein the adhesive material includes medical grade silicone.

10. The system of claim 1, wherein a surface of the bead of adhesive material includes a concave indentation therein.

11. The system of claim 10, wherein the indentation conforms to a surface of the contact lens.

12. A method comprising:
adhering a positioning member to a contact lens using a bead of adhesive material;
maintaining the adherence of the positioning member to the contact lens while the contact lens is positioned onto an eye; and
disengaging the positioning member from the contact lens in response to application of at least a tapping motion with the positioning member.

13. The method of claim 12, wherein the positioning member is further disengaged from the contact lens in response to a blink of the eye.

14. The method of claim 12, further comprising:
supporting the bead of adhesive at a support structure of the positioning member while the contact lens is positioned onto the eye, the support structure encased within the bead of adhesive material.

15. The method of claim 12, further comprising:
receiving the contact lens into a concave indentation in a surface of the bead of adhesive material.

16. The method of claim 12, further comprising:
re-adhering the positioning member to the contact lens using the bead of soft wax while the contact lens is positioned on the eye;
maintaining the adherence of the positioning member to the contact lens while the contact lens is removed from the eye; and
disengaging the positioning member from the contact lens.

17. The method of claim 16, where the positioning member is re-adhered to the positioning member in response to a tap of the bead of adhesive material to an edge of the contact lens, and wherein the contact lens is removed in response to being slid and turned simultaneously by the positioning member.

18. A method comprising:
forming a positioning member with a support structure having a bulb shape;
forming a bead of adhesive material on the positioning member, the bead of adhesive material encasing the bulb shape; and
forming an indentation in a surface of the bead of adhesive material.

19. The method of claim 18, further comprising:
forming a porous texture on the support structure before forming the bead of adhesive material on the positioning member.

20. The method of claim 18, wherein the adhesive material is formed from a wax material, a gel material, medical grade silicone, or a combination thereof.
